(19) United States
(12) Patent Application Publication
(10) Pub. No.: US 2026/0013821 A1
(43) Pub. Date: Jan. 15, 2026

(54) CONTROL APPARATUS, METHOD, AND PROGRAM OF RADIOGRAPHIC IMAGING APPARATUS

(71) Applicant: FUJIFILM Corporation, Tokyo (JP)

(72) Inventors: Shinichi KOJIMA, Tokyo (JP); Isao TAKAHASHI, Tokyo (JP); Kazuma YOKOI, Tokyo (JP)

(73) Assignee: FUJIFILM Corporation, Tokyo (JP)

(21) Appl. No.: 19/263,005

(22) Filed: Jul. 8, 2025

(30) Foreign Application Priority Data
Jul. 12, 2024 (JP) 2024-112940

(51) Int. Cl. A61B 6/00; A61B 6/12; A61B 6/42; A61B 6/50

(52) U.S. Cl. CPC A61B 6/542; A61B 6/12; A61B 6/4241; A61B 6/50

(57) ABSTRACT

In a case of controlling a radiographic imaging apparatus including a photon-counting detector that detects radiation emitted from a radiation source and transmitted through a subject on a patient table and that outputs a detection signal corresponding to a photon energy of the radiation, a high-sensitivity site in the subject, where sensitivity to the radiation is relatively higher than in other sites, is specified based on an optical image of the subject acquired by a camera that acquires the optical image, and different parameters related to a dose of radiation to be emitted to the subject are set between the high-sensitivity site and the other sites.

### CROSS REFERENCE TO RELATED APPLICATIONS

The present application claims priority from Japanese Patent Application No. 2024-112940, filed on Jul. 12, 2024, the entire disclosure of which is incorporated herein by reference.

### BACKGROUND

### Technical Field

The present disclosure relates to a control apparatus, method, and program of a radiographic imaging apparatus.

### Related Art

In recent years, a photon-counting computed tomography (PCCT) apparatus, which is a radiographic imaging apparatus equipped with a photon-counting detector, is known. The PCCT apparatus can obtain a high-resolution image, which is a tomographic image with a higher resolution as compared to a conventional computed tomography (CT) apparatus, and can acquire energy information of each of a plurality of energy bands by measuring the energy for each photon. Therefore, the PCCT apparatus can obtain more information as compared to the conventional CT apparatus.

Meanwhile, a detection signal output by the photon-counting detector has nonlinearity with respect to incident radiation. For this reason, in the PCCT apparatus using the photon-counting detector, it is necessary to set various parameters, such as a dose of radiation and an imaging range, in order to mitigate nonlinearity in a case of imaging a subject. Therefore, a scanogram image is acquired through preliminary imaging, and various parameters are set based on the scanogram image to perform imaging of the subject.

In addition, a method of installing a camera that acquires an optical image of the subject on the PCCT apparatus and using an image acquired by the camera to set parameters during imaging by the PCCT apparatus has been proposed. For example, JP2022-100631A describes an invention that estimates a physique, a transmission length, and an attenuation rate of a subject from an image acquired by a camera to calculate an estimated count number of X-rays in a case of imaging the subject and that determines the number of bits in a case of transmitting detection data detected by a photon-counting detector using the estimated count number. Further, in JP2018-138126A, a method of measuring a size of a subject from an image acquired by a camera and determining a filter to be used for imaging based on the size has been proposed.

Meanwhile, sensitivity to radiation varies depending on the site of the human body. Therefore, in a case where radiation is emitted to the entire body of the subject using the same parameters, sites with higher sensitivity to radiation may be adversely affected by exposure.

### SUMMARY OF THE INVENTION

The present disclosure has been made in view of the above-described circumstances, and an object of the present disclosure is to enable appropriate setting of parameters during imaging according to a site of a subject.

According to the present disclosure, there is provided a control apparatus of a radiographic imaging apparatus including a photon-counting detector that detects radiation emitted from a radiation source and transmitted through a subject on a patient table and that outputs a detection signal corresponding to a photon energy of the radiation, the control apparatus comprising: a processor, in which the processor is configured to: specify a high-sensitivity site in the subject, where sensitivity to the radiation is relatively higher than in other sites, based on an optical image of the subject acquired by a camera that acquires the optical image; and set different parameters related to a dose of radiation to be emitted to the subject between the high-sensitivity site and the other sites.

In the control apparatus of a radiographic imaging apparatus according to the present disclosure, the processor may be configured to set the parameters such that the dose of radiation to the high-sensitivity site is smaller than that to the other sites.

In addition, in the control apparatus of a radiographic imaging apparatus according to the present disclosure, the processor may be configured to: determine whether or not an object other than the subject is included in the optical image; and in a case where the object is included, set a parameter related to the dose of radiation to be transmitted through the object based on information on at least one of a size of the object, a composition of the object, whether or not the object moves together with the patient table, or imaging conditions including an imaging range and a movement speed of the patient table.

Further, in the control apparatus of a radiographic imaging apparatus according to the present disclosure, the processor may be configured to: derive the size of the object based on the optical image; and acquire information on at least one of the composition of the object, whether or not the object moves together with the patient table, or the imaging conditions, based on an input from a user.

Additionally, in the control apparatus of a radiographic imaging apparatus according to the present disclosure, the processor may be configured to acquire information on at least one of the size of the object, the composition of the object, or whether or not the object moves together with the patient table, based on the optical image.

In addition, in the control apparatus of a radiographic imaging apparatus according to the present disclosure, the processor may be configured to issue a notification of the parameter.

Additionally, in the control apparatus of a radiographic imaging apparatus according to the present disclosure, the processor may be configured to: specify, based on the optical image, a non-transmission region in the photon-counting detector, which is a region other than a transmission region where the radiation that has been transmitted through an object including the subject is emitted, according to a projection angle of the radiation with respect to the subject; and perform non-linear correction on a detection signal output from a detection element of the non-transmission region in the photon-counting detector.

Further, in the control apparatus of a radiographic imaging apparatus according to the present disclosure, the processor may be configured to: specify, based on the optical image, a surface layer position of the subject according to the projection angle of the radiation with respect to the subject; and specify the non-transmission region in the photon-counting detector according to the projection angle of the radiation with respect to the subject, based on information on at least one of the surface layer position of the subject, a positional relationship between the subject and the photon-counting detector, an imaging range of the subject, a movement speed of the patient table, or a size of the patient table.

Moreover, in the control apparatus of a radiographic imaging apparatus according to the present disclosure, the processor may be configured to perform the non-linear correction on detection signals output from a predetermined number of detection elements located in the transmission region and adjacent to the non-transmission region.

According to the present disclosure, there is provided a control method of a radiographic imaging apparatus including a photon-counting detector that detects radiation emitted from a radiation source and transmitted through a subject on a patient table and that outputs a detection signal corresponding to a photon energy of the radiation, the control method comprising: causing a computer to: specify a high-sensitivity site in the subject, where sensitivity to the radiation is relatively higher than in other sites, based on an optical image of the subject acquired by a camera that acquires the optical image; and set different parameters related to a dose of radiation to be emitted to the subject between the high-sensitivity site and the other sites.

According to the present disclosure, there is provided a control program of a radiographic imaging apparatus including a photon-counting detector that detects radiation emitted from a radiation source and transmitted through a subject on a patient table and that outputs a detection signal corresponding to a photon energy of the radiation, the control program causing a computer to execute: a procedure of specifying a high-sensitivity site in the subject, where sensitivity to the radiation is relatively higher than in other sites, based on an optical image of the subject acquired by a camera that acquires the optical image; and a procedure of setting different parameters related to a dose of radiation to be emitted to the subject between the high-sensitivity site and the other sites.

The present disclosure can also be applied to a control program product.

According to the present disclosure, it is possible to perform appropriate setting of parameters during imaging according to a site of a subject.

### BRIEF DESCRIPTION OF THE DRAWINGS

FIG. 1 is a schematic configuration diagram of a medical image capturing system comprising a control apparatus of a radiographic imaging apparatus according to a first embodiment of the present disclosure.

FIG. 2 is a diagram showing a hardware configuration of the control apparatus according to the first embodiment.

FIG. 3 is a diagram showing a functional configuration of the control apparatus according to the first embodiment.

FIG. 4 is a diagram illustrating a range in which different parameters are set in a subject.

FIG. 5 is a flowchart showing processing performed in the first embodiment.

FIG. 6 is a diagram showing a notification screen in a case where an external object is included in the optical image.

FIG. 7 is a diagram illustrating a range in which different parameters are set in the subject.

FIG. 8 is a diagram showing a parameter notification screen.

FIG. 9 is a flowchart showing processing performed in a second embodiment.

FIG. 10 is a diagram showing a functional configuration of a control apparatus according to a third embodiment.

FIG. 11 is a diagram illustrating specification of a transmission region.

FIG. 12 is a flowchart showing processing performed in the third embodiment.

### DETAILED DESCRIPTION

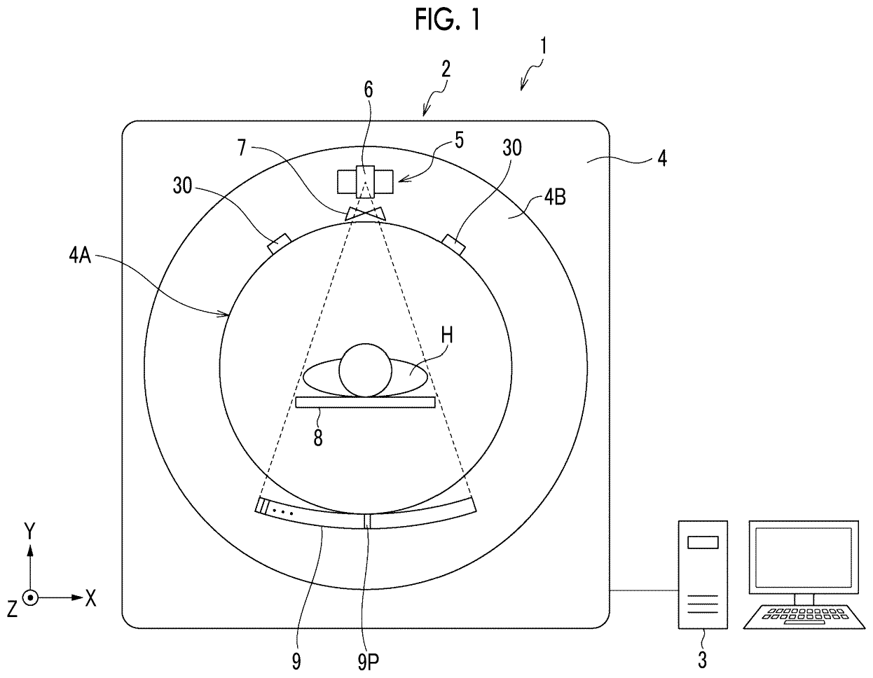

Embodiments of the present disclosure will be described in detail below with reference to the drawings. First, an example of a configuration of a medical image capturing system comprising a control apparatus of a radiographic imaging apparatus according to a first embodiment of the present disclosure will be described. FIG. 1 is a schematic configuration diagram of the medical image capturing system comprising the control apparatus of the radiographic imaging apparatus according to the first embodiment.

As shown in FIG. 1, a medical image capturing system 1 of the present embodiment comprises a CT apparatus 2 and a console 3. The CT apparatus 2 comprises a gantry 4 and a patient table 8. In the following description, a horizontal direction in FIG. 1 is defined as an X axis, a vertical direction is defined as a Y axis, and a direction orthogonal to an XY plane is defined as a Z axis. The CT apparatus 2 is an example of the radiographic imaging apparatus of the present disclosure.

The gantry 4 includes an opening portion 4A, and a subject H to be imaged is disposed in the opening portion 4A in a state of being placed on the patient table 8. The gantry 4 and the patient table 8 are movable relative to each other in a Z-axis direction.

Inside the gantry 4, a radiation source 5 including a radiation tube 6 and a bowtie filter 7 and a detector 9 are disposed to face each other with the subject H interposed therebetween. The bowtie filter 7 optimizes an exposure dose by increasing the dose near the center and reducing the dose around the periphery in order to reduce the exposure dose in a peripheral portion. Radiation emitted from the radiation tube 6 is shaped into a beam shape suitable for a size of the subject H by the bowtie filter 7 and is emitted to the subject H. The detector 9 detects radiation, which has been transmitted through the subject H, and generates projection data corresponding to the dose of the detected radiation. As an example, the detector 9 of the present embodiment is a photon-counting detector in which a plurality of detection elements 9P, which detect a photon energy that is an energy of photons of incident radiation, are disposed in an arc shape centered on a focal point of the radiation tube 6. The detector 9 outputs the projection data corresponding to the photon energy. The pixel value of each pixel position of the projection data is a value of a detection signal output from each of the detection elements 9P.

In the present embodiment, X-rays are used as an example of the radiation, but the present disclosure is not limited to this.

The radiation tube 6 and the detector 9 are attached to a rotating plate 4B in the gantry 4 and are rotated around the subject H by a rotation driving unit (not shown). The radiation irradiation from the radiation tube 6 and the detection of the radiation by the detector 9 are repeated with the rotation of the radiation tube 6 and the detector 9, thereby acquiring projection data at various projection angles. A plurality of pieces of projection data acquired by the detector 9 are output to the console 3.

The dose of radiation emitted from the radiation tube 6, a rotation speed of the gantry 4, a relative movement speed between the gantry 4 and the patient table 8, and the like are set by the console 3 based on acquisition conditions for acquiring projection data, which are input from a user, such as a technician.

In addition, two cameras 30 are installed on the gantry 4 to face the opening portion 4A. The camera 30 is, for example, a camera capable of capturing an RGB color optical image by detecting reflected light from the subject H. The camera 30 includes a lens and an imaging element, such as a charge coupled device (CCD), acquires an optical image G0 by imaging the subject H on the patient table 8, and outputs the optical image G0 to the console 3. The camera 30 may acquire a monochrome image. Further, the number of cameras 30 is two, but may be one, or three or more.

In a case where the subject His imaged by the camera 30, by moving the patient table 8 in a Z direction, the optical image G0 is acquired so as to include an imaging site of the subject H. In a case where the entire imaging site of the subject H is not included in the optical image G0 in a single imaging operation by the camera 30, a plurality of imaging operations need only be performed by the camera 30, and the optical images G0 acquired by the plurality of imaging operations need only be stitched together to acquire the optical image G0 including the entire imaging site of the subject H.

The console 3 of the first embodiment performs control related to acquisition of projection data through imaging of the subject H, generation of a tomographic image from the projection data, setting of parameters to be described below using the optical image G0, and the like. The console 3 is an example of a control apparatus of the present disclosure.

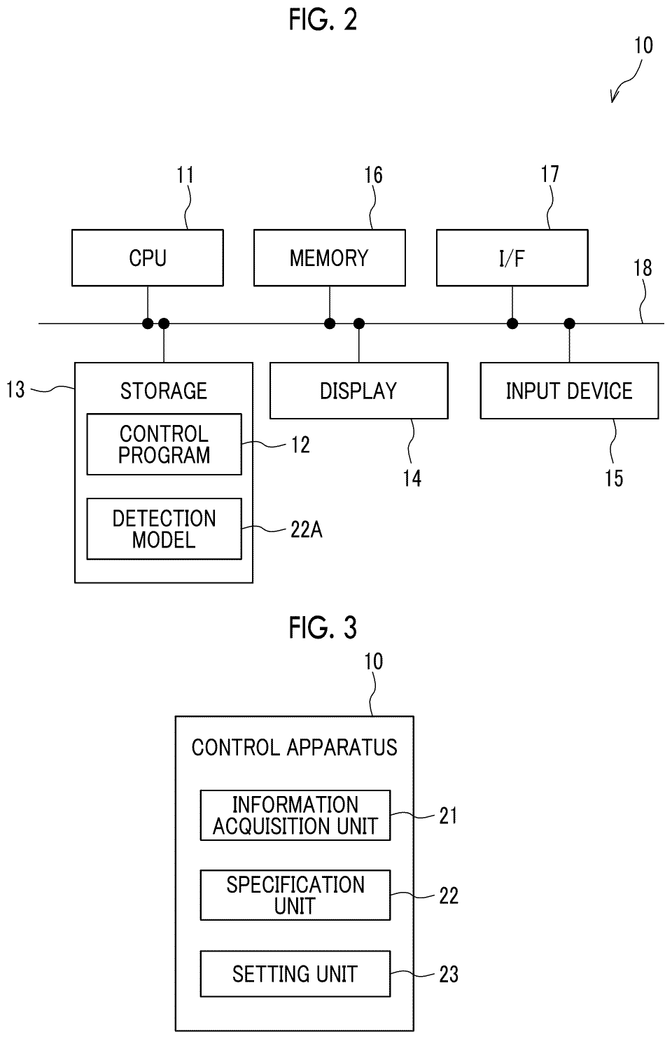

Next, the control apparatus according to the first embodiment will be described. First, a hardware configuration of the control apparatus according to the first embodiment, which is incorporated into the console 3, will be described with reference to FIG. 2. As shown in FIG. 2, a control apparatus 10, which is incorporated into the console 3, is a computer, such as a workstation, a server computer, or a personal computer, and comprises a central processing unit (CPU) 11, a non-volatile storage 13, and a memory 16 as a temporary storage area.

Additionally, the control apparatus 10 comprises a display 14, an input device 15, and an interface (I/F) 17. The CPU 11, the storage 13, the display 14, the input device 15, the memory 16, and the I/F 17 are connected to a bus 18. The CPU 11 is an example of a processor in the present disclosure.

The storage 13 is implemented by a hard disk drive (HDD), a solid state drive (SSD), a flash memory, or the like. The storage 13 as a storage medium stores a control program 12 installed in the control apparatus 10. The CPU 11 reads out the control program 12 from the storage 13, loads the control program 12 onto the memory 16, and executes the loaded control program 12.

The display 14 is a device that displays various screens and is, for example, a liquid crystal display or an electro luminescence (EL) display.

The input device 15 is used by the user to input acquisition conditions for acquiring the projection data, instructions or various kinds of information related to generation and display of images, and the like. Examples of the input device 15 include various switches, buttons, a touch panel, a touch pen, a keyboard, and a mouse. The display 14 and the input device 15 may be integrated into a touch panel display.

The I/F 17 performs communication of various kinds of information with the rotation driving unit (not shown) of the gantry 4, the radiation source 5, the detector 9, and the camera 30 through wired communication or wireless communication.

The control program 12 is stored in a storage device of a server computer connected to a network or in a network storage in a state of being accessible from the outside and is downloaded and installed on the computer that constitutes the control apparatus 10 in response to the request. Alternatively, the control program 12 is distributed by being recorded on a recording medium, such as a digital versatile disc (DVD) or a compact disc read only memory (CD-ROM), and is installed on the computer that constitutes the control apparatus 10 from the recording medium.

Next, a functional configuration of the control apparatus according to the first embodiment will be described. FIG. 3 is a diagram showing the functional configuration of the control apparatus according to the first embodiment. As shown in FIG. 3, the control apparatus 10 comprises an information acquisition unit 21, a specification unit 22, and a setting unit 23. The CPU 11 executes the control program 12 to function as the information acquisition unit 21, the specification unit 22, and the setting unit 23.

The information acquisition unit 21 acquires the optical image G0, which is acquired by the camera 30 of the CT apparatus 2 through imaging of the subject H, via the I/F 17.

The specification unit 22 specifies a high-sensitivity site in the subject H, where sensitivity to radiation is relatively higher than other sites, based on the optical image G0. In the present embodiment, the high-sensitivity site is the eyes of the subject H. In the present embodiment, a detection model 22A constructed by machine learning a neural network to detect the eyes of the subject H from the optical image G0 is stored in the storage 13. The specification unit 22 specifies the position of the eyes of the subject H in the optical image G0 using the detection model 22A. Further, the specification unit 22 specifies the position of the eyes of the subject H on the patient table 8 in the Z direction based on the optical image G0.

In addition, in the present embodiment, the average size and the variance σ of the eyeball are derived based on tomographic images obtained from projection data acquired by the CT apparatus 2 for a large number of subjects H, a size range of the eyeball is derived, for example, as a range of 30 with respect to the average size of the eyeball, and the size range is stored in the storage 13.

The specification unit 22 specifies the size range of the eyeball based on the position of the eyes in the Z direction of the patient table 8, as a range of the eyes in which the dose of radiation is reduced.

The setting unit 23 sets different parameters related to the dose of radiation to be emitted to the subject H between the eyes, which are a high-sensitivity site of the subject H, and other sites other than the eyes. Specifically, a tube current with respect to the radiation source 5 is set such that the dose of radiation to the high-sensitivity site is, for example, about 10% lower than that to the other sites. It should be noted that the extent to which the dose of radiation is reduced is not limited to 10%.

Additionally, since a tube voltage also affects the dose of radiation, the setting unit 23 may set the tube voltage in addition to the tube current or instead of the tube current. The tube current and the tube voltage are examples of parameters in the present disclosure. Further, in the present embodiment, making the parameters different includes not only setting, for example, different parameter values, such as a value of a tube current and a value of a tube voltage, but also setting different types of parameters, such as a tube current and a tube voltage. In the following description, it is assumed that the setting unit 23 sets the type of parameter as the tube current, sets the parameter related to the dose of radiation for sites other than the eyes as a first tube current, and sets the parameter related to the dose of radiation for the eyes as a second tube current smaller than the first tube current.

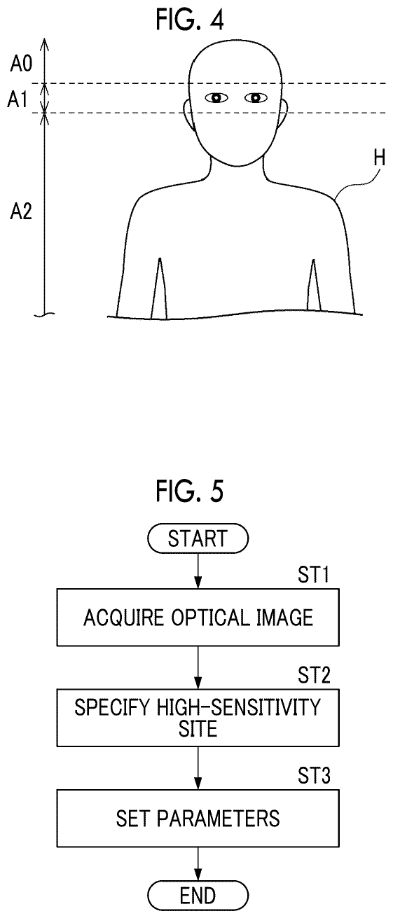

Here, in the CT apparatus 2, the subject H is imaged at various projection angles by rotating the radiation source 5 and the detector 9 while the patient table 8 is moved in the Z direction. Therefore, as the patient table 8 moves in the Z direction, the irradiation position of the radiation on the subject H moves. FIG. 4 is a diagram illustrating the irradiation position of the radiation on the subject H. Since the radiation to be emitted from the radiation source 5 spreads in the Z direction of the patient table 8, the subject H is irradiated with radiation within a certain irradiation range. The irradiation range of the radiation in the X direction shown in FIG. 1 matches a range of the detector 9 in a channel direction. Meanwhile, the irradiation range of the radiation in the Z direction varies depending on the size of the subject H, but is, for example, about 40 mm to 160 mm in the vicinity of the center of the irradiation range.

The setting unit 23 specifies a timing at which the range of the eyes of the subject H enters the irradiation range of the radiation and a timing at which the range of the eyes of the subject H exits the irradiation range after the start of imaging, based on the initial position of the patient table 8, the position of the eyes in the Z direction of the patient table 8, the movement speed of the patient table 8 in the Z direction, and the irradiation range of the radiation in the Z direction.

Then, the setting unit 23 drives the radiation source 5 with the first tube current from the start of imaging until the timing at which the range of the eyes of the subject H enters the irradiation range of the radiation. The setting unit 23 drives the radiation source 5 with the second tube current from the timing at which the range of the eyes of the subject H enters the irradiation range of the radiation until the timing at which the range of the eyes of the subject H exits the irradiation range of the radiation. Then, the setting unit 23 drives the radiation source 5 with the first tube current from the timing at which the range of the eyes of the subject H exits the irradiation range of the radiation until the end of imaging.

Consequently, as shown in FIG. 4, a range A0 and a range A2 in the subject H other than a range A1 of the eyes are irradiated with radiation having a dose based on the first tube current. Meanwhile, the range A1 of the eyes in the subject H is irradiated with radiation having a dose based on the second tube current smaller than the first tube current.

Next, the processing performed in the first embodiment will be described. FIG. 5 is a flowchart showing the processing performed in the first embodiment. First, the information acquisition unit 21 acquires the optical image G0 acquired by the camera 30 through imaging of the subject H (step ST1). Next, the specification unit 22 specifies the high-sensitivity site of the subject H (step ST2). Then, the setting unit 23 sets different parameters related to the dose of radiation to be emitted to the subject H between the high-sensitivity site and the other sites (step ST3), and the processing ends.

After that, the subject His imaged using the set parameters to acquire the projection data. In addition, the tomographic image is derived by reconstructing the projection data.

In this way, in the first embodiment, different parameters related to the dose of radiation to be emitted to the subject H are set between the high-sensitivity site and other sites in the subject H. Therefore, the parameters during imaging can be appropriately set according to the site of the subject.

In particular, in the first embodiment, the parameters are set such that the dose of radiation to the high-sensitivity site to radiation, such as the eyes, is lower than the dose of radiation to the other sites. Therefore, it is possible to prevent high-dose radiation from being emitted to the high-sensitivity site to radiation, such as the eyes.

Next, a second embodiment of the present disclosure will be described. The configuration of a control apparatus according to the second embodiment is the same as the configuration of the control apparatus according to the first embodiment, and only processing to be performed is different. Therefore, detailed description of the apparatus will be omitted here.

The second embodiment relates to setting of parameters of the dose of radiation in a case where an object (hereinafter, referred to as an external object) other than the subject H is included in the optical image G0. Therefore, in the second embodiment, the specification unit 22 first determines whether or not an external object other than the subject H is included in the optical image G0.

In the second embodiment, a detection model constructed by machine learning a neural network to detect the external object is stored in the storage 13. The specification unit 22 detects the external object from the optical image G0 using the detection model. Additionally, the specification unit 22 specifies a range of the external object on the patient table 8 in the Z direction based on the optical image G0.

In the second embodiment, the setting unit 23 sets the parameter of the dose of radiation to be transmitted through the external object based on information on at least one of a size of the external object, a composition of the external object, whether or not the external object moves together with the patient table 8, or imaging conditions including the imaging range and the movement speed of the patient table 8.

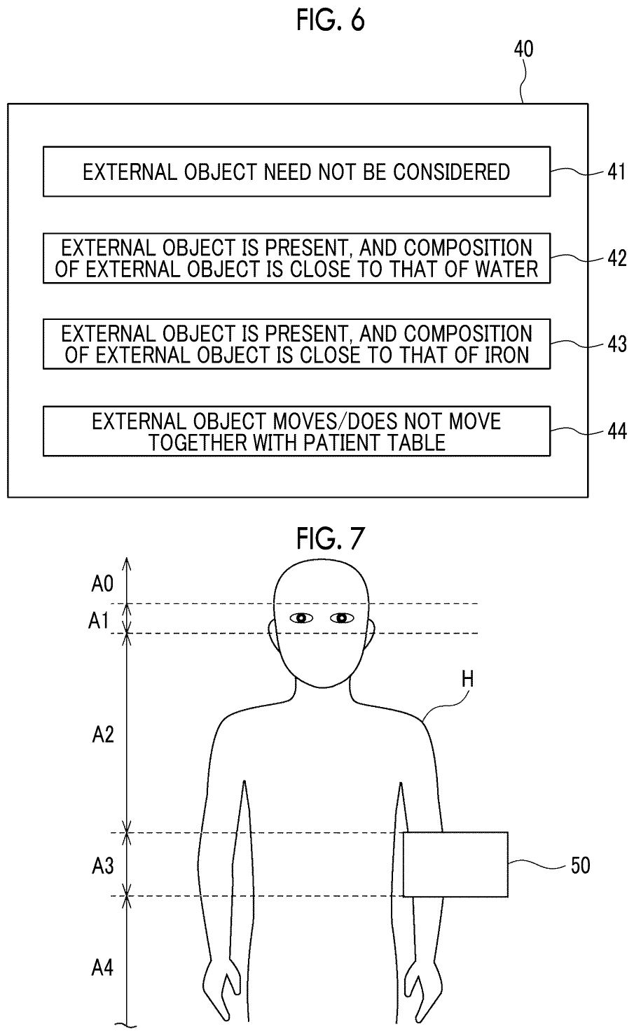

In the second embodiment, in a case where the external object is included in the optical image G0, the specification unit 22 displays a notification screen to the user on the display 14. FIG. 6 is a diagram showing the notification screen in a case where the external object is included in the optical image G0. As shown in FIG. 6, the following options are displayed on a notification screen 40: an option 41 stating “the external object need not be considered”, an option 42 stating “the external object is present, and the composition of the external object is close to that of water”, an option 43 stating “the external object is present, and the composition of the external object is close to that of iron”, and an option 44 stating “the external object moves/does not move together with the patient table”.

First, the user selects a desired option among the options 41 to 43. In a case where the option 42 or 43 is selected, the user further selects whether or not the external object moves together with the patient table 8 in the option 44. The imaging conditions are input to the console 3 by the user in advance in a case where the subject His imaged.

Examples of the external object that moves together with the patient table 8 and that has a composition close to that of water include a patient table, a pillow, and a blanket, which are made largely of carbon-based materials. Examples of the external object that does not move together with the patient table 8 and that has a composition close to that of water include an intravenous drip tube that lies outside the irradiation range of the radiation, expands and contracts with the movement of the patient table 8, but moves differently from the movement of the patient table 8. Examples of the external object that moves together with the patient table 8 and that has a composition close to that of iron include an injection needle for a contrast agent and an intravenous drip, a head fixation device, and a contrast agent injector. In addition, with regard to the external object that does not move together with the patient table 8 and that has a composition close to that of iron, such an external object is assumed not to be present because unnecessary metal is not placed within the irradiation range of the radiation during imaging.

Here, in the CT apparatus 2, the subject H is imaged by rotating the radiation source 5 and the detector 9 while moving the patient table 8 in the Z direction. Therefore, as the patient table 8 moves in the Z direction, the irradiation range of the radiation on the subject H moves. Additionally, as mentioned above, the irradiation range of the radiation in the Z direction is, for example, about 40 mm to 160 mm in the vicinity of the center of the irradiation range. The setting unit 23 specifies a timing at which the range of the eyes of the subject H and the range of the external object enter the irradiation range of the radiation and a timing at which the range of the eyes of the subject H and the range of the external object exit the irradiation range after the start of imaging, based on the initial position of the patient table 8, the position of the eyes in the Z direction of the patient table 8, the range of the external object in the Z direction of the patient table 8, the movement speed of the patient table 8 in the Z direction, and the irradiation range of the radiation in the Z direction.

The setting unit 23 drives the radiation source 5 with the first tube current from the start of imaging until the timing at which the range of the eyes of the subject H enters the irradiation range of the radiation, in the same manner as in the first embodiment. The setting unit 23 drives the radiation source 5 with the second tube current from the timing at which the range of the eyes of the subject H enters the irradiation range of the radiation until the timing at which the range of the eyes of the subject H exits the irradiation range of the radiation. Then, the setting unit 23 drives the radiation source 5 with the first tube current from the timing at which the range of the eyes of the subject H exits the irradiation range of the radiation until the end of imaging.

In addition, the setting unit 23 drives the radiation source 5 with the first tube current from the start of imaging until the timing at which the range of the external object enters the irradiation range of the radiation, except for a period during which the range of the eyes of the subject H is within the irradiation range of the radiation. The setting unit 23 drives the radiation source 5 with a third tube current from the timing at which the range of the external object enters the irradiation range of the radiation until the timing at which the range of the external object exits the irradiation range of the radiation. Then, the setting unit 23 drives the radiation source 5 with the first tube current from the timing at which the range of the external object exits the irradiation range of the radiation until the end of imaging.

The third tube current need only be set by the user according to the composition of the external object and the transmission distance of the radiation in the external object. For example, in a case where the composition of the external object is close to that of water and the transmission distance of the radiation is, for example, 5 cm or less, the third tube current need only be set to be the same as the first tube current. In a case where the composition of the external object is close to that of water and the transmission distance of the radiation exceeds, for example, 5 cm, and in a case where the composition of the external object is close to that of iron, the third tube current larger than the first tube current need only be set to increase the dose of radiation.

Consequently, as shown in FIG. 7, ranges A0, A2, and A4 in the subject H other than the range A1 of the eyes and a range A3 of an external object 50 are irradiated with radiation having a dose based on the first tube current. Meanwhile, the range A1 of the eyes in the subject His irradiated with radiation having a dose based on the second tube current smaller than the first tube current. Additionally, the range A3 of the external object 50 is irradiated with radiation having a dose based on the third tube current.

The setting unit 23 may acquire information on the size of the external object, the composition of the external object, and whether or not the external object moves together with the patient table 8 based on the optical image G0, determine the composition of the external object, and automatically set the parameters based on the acquired information and the determined result.

For example, in a case where the external object 50 detected from the optical image G0 has a length of 50 cm or greater and a width of 20 cm or greater, and the external object 50 is present on the subject H, the setting unit 23 may determine that the external object 50 is a blanket and the composition is, for example, polyester. In addition, in a case where the external object 50 detected from the optical image G0 is located beside the head of the subject H and has a size of about 5 cm square, the setting unit 23 may determine that the external object 50 is the head fixation device and the composition is iron.

In this case, the setting unit 23 sets the parameter, that is, the third tube current, according to the determined composition of the external object and the transmission distance of the radiation. For example, in a case where the composition of the external object 50 is close to that of water, such as polyester, and the transmission distance of the radiation is, for example, 5 cm or less, the third tube current need only be set to be the same as the first tube current, in the same manner as in a case where the operator performs the settings. In a case where the composition of the external object is close to that of water and the transmission distance of the radiation exceeds, for example, 5 cm, and in a case where the composition of the external object is close to that of iron, the third tube current larger than the first tube current need only be set to increase the dose of radiation.

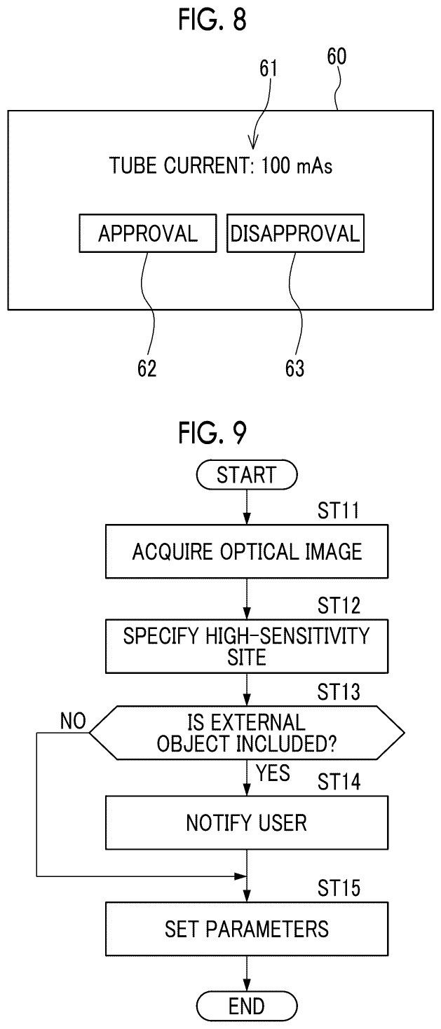

Increasing the dose of radiation results in a higher exposure dose to the subject H. Therefore, the setting unit 23 may notify the user of the set third tube current, that is, the parameter, through display on the display 14 or the like before imaging. FIG. 8 is a diagram showing a parameter notification screen. As shown in FIG. 8, a value 61 of the third tube current and buttons 62 and 63 for approval and disapproval instructions are displayed on a parameter notification screen 60. In a case where the user selects the approval button 62, an imaging instruction is issued from the console 3 to the CT apparatus 2, and imaging is performed. In a case where the disapproval button 63 is selected, for example, a setting screen for the user to set the parameters through a manual operation is displayed on the display 14. The user sets the parameters through a manual operation on the setting screen.

Next, the processing performed in the second embodiment will be described. FIG. 9 is a flowchart showing the processing performed in the second embodiment. First, the information acquisition unit 21 acquires the optical image G0 acquired by the camera 30 through imaging of the subject H (step ST11). Next, the specification unit 22 specifies the high-sensitivity site of the subject H (step ST12). Additionally, in the second embodiment, the specification unit 22 determines whether or not the external object is included in the optical image G0 (step ST13).

In a case where an affirmative determination is made in step ST13, the setting unit 23 displays the notification screen shown in FIG. 6 on the display 14 (notification to the user, step ST14). In a case where a negative determination is made in step ST13, the processing transitions to step ST15. Then, the setting unit 23 sets different parameters related to the dose of radiation to be emitted to the subject H between the high-sensitivity site and other sites, as well as between the region of the external object and other regions, based on the input from the user (step ST15), and the processing ends.

After that, the subject His imaged using the set parameters to acquire the projection data. In addition, the tomographic image is derived by reconstructing the projection data.

In this way, in the second embodiment, in a case where the external object is included in the optical image G0, the parameters are set in consideration of the external object. Therefore, the parameters during imaging can be appropriately set in consideration of the dose to the external object.

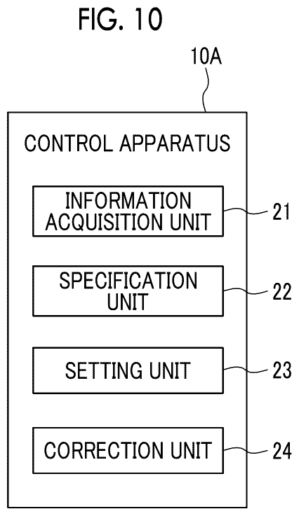

Next, a third embodiment of the present disclosure will be described. FIG. 10 is a diagram showing a functional configuration of a control apparatus according to the third embodiment. In FIG. 10, the same configurations as those in FIG. 3 are assigned the same reference numerals, and detailed descriptions thereof will be omitted here. A control apparatus 10A according to the third embodiment is different from that of the first embodiment in that a correction unit 24 is further provided.

In the third embodiment, the specification unit 22 specifies, based on the optical image G0, a non-transmission region in the detector 9, which is a region other than a transmission region irradiated with radiation that has been transmitted through an object including the subject H and which is directly irradiated with radiation, in accordance with the projection angle of the radiation with respect to the subject H. Then, the correction unit 24 performs non-linear correction on the detection signal output from the detection element in the non-transmission region in the detector 9.

In the third embodiment, the specification unit 22 specifies a surface layer position of the subject H in the optical image G0 in accordance with the projection angle of the radiation with respect to the subject H. Therefore, in the third embodiment, a learning model constructed by machine learning a neural network to specify the surface layer position of the subject H from the optical image G0 is stored in the storage 13. The specification unit 22 specifies the surface layer position of the subject H in the optical image G0 using the learning model.

Here, in the CT apparatus 2, the radiation tube 6 and the detector 9 rotate around the subject H to image the subject H at various projection angles of radiation. Therefore, the direction in which the subject H is irradiated with the radiation differs depending on the projection angle.

For example, as shown in FIG. 1, in a projection angle at which radiation is emitted to the front surface of the subject H, the range of the radiation emitted from the radiation source 5 passes through an end portion of the patient table 8 including the surface layer position of the subject H. Therefore, at the projection angle shown in FIG. 1, there is no non-transmission region in the detector 9.

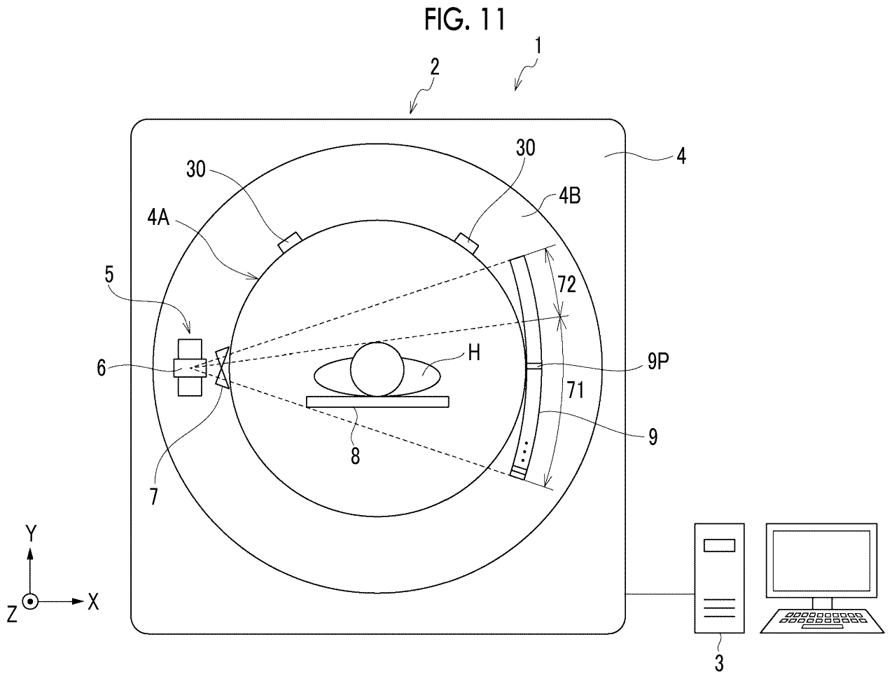

On the other hand, as shown in FIG. 11, in a projection angle at which radiation is emitted to the side surface of the subject H, the radiation emitted from the radiation source 5 includes a transmitted portion that passes through the surface layer position of the subject H and the patient table 8, and a non-transmitted portion. Therefore, at the projection angle shown in FIG. 11, in the detector 9, there are a transmission region 71 where the radiation that has been transmitted through the subject H and the patient table 8 is incident and a non-transmission region 72 where the radiation is directly incident without being transmitted through the subject H and the patient table 8.

In the third embodiment, the specification unit 22 specifies, according to the projection angle, the transmission region 71 and the non-transmission region 72, based on at least one of the surface layer position of the subject H, the positional relationship between the detector 9 and the subject H, information such as the imaging conditions including the imaging range set by the user and the movement speed of the patient table 8, or information on the size of the patient table 8.

Specifically, the specification unit 22 specifies a body axis direction of the subject H on the patient table 8, that is, the imaging range in the Z direction on the patient table 8, based on the surface layer position of the subject H and the imaging range set by the user. A position where the subject H passes through the irradiation range of the radiation is specified according to the initial position of the patient table 8 and the movement speed of the patient table 8. Additionally, the positional relationship between the detector 9 and the subject H is specified according to the projection angle and the movement speed of the patient table 8. Further, the irradiation range of the radiation in the Z direction and the irradiation range of the detector 9 in the channel direction are known as mentioned above. Therefore, it is possible to specify the positional relationship between the subject H and the patient table 8, and the detector 9, and the irradiation range of the radiation at each of the projection angles during imaging. The specification unit 22 specifies the transmission region 71 and the non-transmission region 72 on the detector 9 at each of the projection angles based on the specified result. As a result, it is possible to specify whether each of the plurality of detection elements provided in the detector 9 is located in the transmission region 71 or the non-transmission region 72 at each of the projection angles during imaging.

In the projection angle of the radiation shown in FIG. 1, there is no non-transmission region 72. Therefore, the specification unit 22 may determine whether or not the transmission region 71 is present in the detector 9 according to the projection angle, and then specify the transmission region 71 and the non-transmission region 72.

In a case where the non-transmission region 72 is present in the detector 9, the correction unit 24 performs non-linear correction on the detection signal output from the detection element in the non-transmission region 72. In the present embodiment, in the CT apparatus 2, the projection data consisting of the detection signal output from each of the detection elements of the detector 9 is transmitted to the console 3. In the projection data, the pixel value of each pixel is a signal value of the detection signal. Therefore, the correction unit 24 performs non-linear correction on the detection signal at a position corresponding to the non-transmission region 72 in the projection data.

Examples of the non-linear correction include correction for pile-up in which the apparent count value of the photons, that is, the value of the detection signal, increases, and correction for count loss in which the value of the detection signal decreases, but the non-linear correction is not limited to these. Specifically, examples of the non-linear correction include a method of modeling and formulating pile-up and count loss and performing correction using the formulated equations. In addition, examples of the non-linear correction include a method in which a change in signal values output when the tube current is varied is empirically measured, correction data is obtained in advance based on the empirically measured result, and correction is performed using the correction data. The correction unit 24 does not perform non-linear correction on the detection signal output by the detection element in the transmission region 71. Meanwhile, at the boundary between the transmission region 71 and the non-transmission region 72 in the projection data, there may be cases where the detection signals for several pixels may not actually be transmitted through the subject H and the patient table 8. Therefore, non-linear correction may be performed on the detection signals output from a predetermined number of detection elements located in the transmission region 71 and adjacent to the non-transmission region 72. In this case, the degree of non-linear correction may be gradually reduced from the boundary with the non-transmission region 72 toward the inside of the transmission region. As a result, it is possible to suppress the occurrence of discontinuities in pixel values due to non-linear correction at the boundary between the transmission region 71 and the non-transmission region 72.

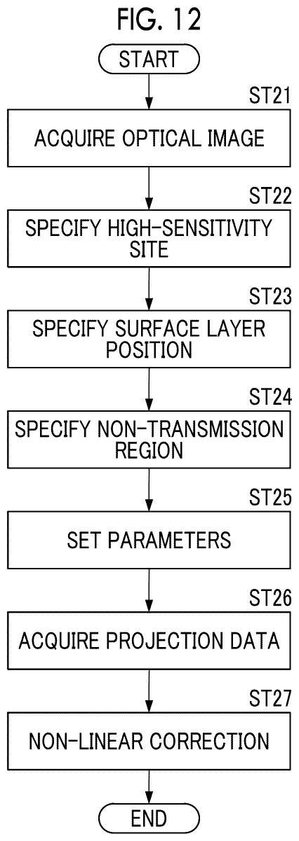

Next, the processing performed in the third embodiment will be described. FIG. 12 is a flowchart showing the processing performed in the third embodiment. First, the information acquisition unit 21 acquires the optical image G0 acquired by the camera 30 through imaging of the subject H (step ST21). Next, the specification unit 22 specifies the high-sensitivity site of the subject H (step ST22).

In the third embodiment, the specification unit 22 further specifies, based on the optical image G0, the surface layer position of the subject H in accordance with the projection angle of the radiation with respect to the subject H (step ST23). Then, the specification unit 22 specifies the non-transmission region 72 in the detector 9 in accordance with the projection angle of the radiation with respect to the subject H, based on at least one of the surface layer position of the subject H, the positional relationship between the detector 9 and the subject H, information such as the imaging conditions including the imaging range set by the user and the movement speed of the patient table 8, or information on the size of the patient table 8 (step ST24). Then, the setting unit 23 sets different parameters related to the dose of radiation to be emitted to the subject H between the high-sensitivity site and other sites (step ST25).

Subsequently, the projection data is acquired at various projection angles of the radiation using the set parameters (step ST26). Then, the correction unit 24 performs non-linear correction on the detection signal corresponding to the non-transmission region 72 in the projection data (step ST27), and the processing ends.

The projection data subjected to the non-linear correction or the projection data not subjected to the non-linear correction is reconstructed to generate a tomographic image.

In this way, in the third embodiment, the non-transmission region 72 is specified according to the projection angle of the radiation with respect to the subject H, and the non-linear correction is performed on the detection signal of the non-transmission region 72. Therefore, it is possible to acquire projection data in which the influence of the pile-up, the count loss, or the like, which occurs in a case where the radiation is directly emitted to the detector 9, is reduced. Therefore, it is possible to improve the image quality of the tomographic image acquired by reconstructing the projection data.

In the above-described third embodiment, the non-linear correction is performed by specifying the non-transmission region 72 according to the projection angle of the radiation with respect to the subject H in the first embodiment, but the present disclosure is not limited to this. In the second embodiment, the non-linear correction may be performed by specifying the non-transmission region 72 according to the projection angle of the radiation with respect to the subject H.

Additionally, in each of the above-described embodiments, the parameters during imaging are set using the optical image G0, but the present disclosure is not limited to this. A scanogram image may be acquired by preliminarily imaging the subject H, and the parameters during imaging may be set by further using the scanogram image in addition to the optical image G0. As a result, it is possible to set parameters more suitable for imaging the subject H as compared to a case where only the optical image G0 is used.

In addition, in each of the above-described embodiments, the camera is installed on the gantry 4, but the present disclosure is not limited to this. For example, the camera 30 may be installed on a ceiling or the like of an imaging room where the CT apparatus 2 is installed.

Additionally, in each of the above-described embodiments, various types of processors to be described below can be used as the hardware structure of the control apparatus 10. The various processors include, in addition to a CPU which is a general-purpose processor that executes software (programs) to function as various processing units, a programmable logic device (PLD) whose circuit configuration can be changed after manufacturing, such as a field-programmable gate array (FPGA), a dedicated electrical circuit which is a processor having a circuit configuration dedicatedly designed for executing specific processing, such as an ASIC, and the like.

In addition, the above-described various kinds of processing may be executed using one of these various processors or may be executed using a combination of two or more processors of the same type or different types (for example, a plurality of FPGAs, a combination of a CPU and an FPGA, and the like). Alternatively, a plurality of processing units may be configured using one processor. As an example of configuring a plurality of processing units using one processor, there is an aspect in which a processor that implements all functions of a system, including a plurality of processing units, with one integrated circuit (IC) chip is used, as in a system on a chip (SOC) and the like.

Hereinafter, the supplementary claims of the present disclosure will be described.

### Supplementary Claim 1

A control apparatus of a radiographic imaging apparatus including a photon-counting detector that detects radiation emitted from a radiation source and transmitted through a subject on a patient table and that outputs a detection signal corresponding to a photon energy of the radiation, the control apparatus comprising:
a processor,
in which the processor is configured to:
   specify a high-sensitivity site in the subject, where sensitivity to the radiation is relatively higher than in other sites, based on an optical image of the subject acquired by a camera that acquires the optical image; and
   set different parameters related to a dose of radiation to be emitted to the subject between the high-sensitivity site and the other sites.

### Supplementary Claim 2

The control apparatus according to Supplementary claim 1,
in which the processor is configured to set the parameters such that the dose of radiation to the high-sensitivity site is smaller than that to the other sites.

### Supplementary Claim 3

The control apparatus according to Supplementary claim 1 or 2,
in which the processor is configured to:
   determine whether or not an object other than the subject is included in the optical image; and
   in a case where the object is included, set a parameter related to the dose of radiation to be transmitted through the object based on information on at least one of a size of the object, a composition of the object, whether or not the object moves together with the patient table, or imaging conditions including an imaging range and a movement speed of the patient table.

### Supplementary Claim 4

The control apparatus according to Supplementary claim 3,
in which the processor is configured to:
   derive the size of the object based on the optical image; and
   acquire information on at least one of the composition of the object, whether or not the object moves together with the patient table, or the imaging conditions, based on an input from a user.

### Supplementary Claim 5

The control apparatus according to Supplementary claim 3,
in which the processor is configured to acquire information on at least one of the size of the object, the composition of the object, or whether or not the object moves together with the patient table, based on the optical image.

### Supplementary Claim 6

The control apparatus according to any one of Supplementary claims 3 to 5,
in which the processor is configured to issue a notification of the parameter.

### Supplementary Claim 7

The control apparatus according to any one of Supplementary claims 1 to 6,
in which the processor is configured to:
   specify, based on the optical image, a non-transmission region in the photon-counting detector, which is a region other than a transmission region where the radiation that has been transmitted through an object including the subject is emitted, according to a projection angle of the radiation with respect to the subject; and
   perform non-linear correction on a detection signal output from a detection element of the non-transmission region in the photon-counting detector.

### Supplementary Claim 8

The control apparatus according to Supplementary claim 7,
in which the processor is configured to:
   specify, based on the optical image, a surface layer position of the subject according to the projection angle of the radiation with respect to the subject; and
   specify the non-transmission region in the photon-counting detector according to the projection angle of the radiation with respect to the subject, based on information on at least one of the surface layer position of the subject, a positional relationship between the subject and the photon-counting detector, an imaging range of the subject, a movement speed of the patient table, or a size of the patient table.

### Supplementary Claim 9

The control apparatus according to Supplementary claim 7 or 8,
in which the processor is configured to perform the non-linear correction on detection signals output from a predetermined number of detection elements located in the transmission region and adjacent to the non-transmission region.

### Supplementary Claim 10

A control method of a radiographic imaging apparatus including a photon-counting detector that detects radiation emitted from a radiation source and transmitted through a subject on a patient table and that outputs a detection signal corresponding to a photon energy of the radiation, the control method comprising:
causing a computer to:
   specify a high-sensitivity site in the subject, where sensitivity to the radiation is relatively higher than in other sites, based on an optical image of the subject acquired by a camera that acquires the optical image; and
   set different parameters related to a dose of radiation to be emitted to the subject between the high-sensitivity site and the other sites.

### Supplementary Claim 11

A control program of a radiographic imaging apparatus including a photon-counting detector that detects radiation emitted from a radiation source and transmitted through a subject on a patient table and that outputs a detection signal corresponding to a photon energy of the radiation, the control program causing a computer to execute:
a procedure of specifying a high-sensitivity site in the subject, where sensitivity to the radiation is relatively higher than in other sites, based on an optical image of the subject acquired by a camera that acquires the optical image; and
a procedure of setting different parameters related to a dose of radiation to be emitted to the subject between the high-sensitivity site and the other sites.

What is claimed is:

1. A control apparatus of a radiographic imaging apparatus including a photon-counting detector that detects radiation emitted from a radiation source and transmitted through a subject on a patient table and that outputs a detection signal corresponding to a photon energy of the radiation, the control apparatus comprising:
a processor,
wherein the processor is configured to:
specify a high-sensitivity site in the subject, where sensitivity to the radiation is relatively higher than in other sites, based on an optical image of the subject acquired by a camera that acquires the optical image; and
set different parameters related to a dose of radiation to be emitted to the subject between the high-sensitivity site and the other sites.

2. The control apparatus according to claim 1,
wherein the processor is configured to set the parameters such that the dose of radiation to the high-sensitivity site is smaller than that to the other sites.

3. The control apparatus according to claim 1,
wherein the processor is configured to:
determine whether or not an object other than the subject is included in the optical image; and
in a case where the object is included, set a parameter related to the dose of radiation to be transmitted through the object based on information on at least one of a size of the object, a composition of the object, whether or not the object moves together with the patient table, or imaging conditions including an imaging range and a movement speed of the patient table.

4. The control apparatus according to claim 3,
wherein the processor is configured to:
derive the size of the object based on the optical image; and
acquire information on at least one of the composition of the object, whether or not the object moves together with the patient table, or the imaging conditions, based on an input from a user.

5. The control apparatus according to claim 3,
wherein the processor is configured to acquire information on at least one of the size of the object, the composition of the object, or whether or not the object moves together with the patient table, based on the optical image.

6. The control apparatus according to claim 3,
wherein the processor is configured to issue a notification of the parameter.

7. The control apparatus according to claim 1,
wherein the processor is configured to:
specify, based on the optical image, a non-transmission region in the photon-counting detector, which is a region other than a transmission region where the radiation that has been transmitted through an object including the subject is emitted, according to a projection angle of the radiation with respect to the subject; and
perform non-linear correction on a detection signal output from a detection element of the non-transmission region in the photon-counting detector.

8. The control apparatus according to claim 7,
wherein the processor is configured to:
specify, based on the optical image, a surface layer position of the subject according to the projection angle of the radiation with respect to the subject; and
specify the non-transmission region in the photon-counting detector according to the projection angle of the radiation with respect to the subject, based on information on at least one of the surface layer position of the subject, a positional relationship between the subject and the photon-counting detector, an imaging range of the subject, a movement speed of the patient table, or a size of the patient table.

9. The control apparatus according to claim 7,
wherein the processor is configured to perform the non-linear correction on detection signals output from a predetermined number of detection elements located in the transmission region and adjacent to the non-transmission region.

10. A control method of a radiographic imaging apparatus including a photon-counting detector that detects radiation emitted from a radiation source and transmitted through a subject on a patient table and that outputs a detection signal corresponding to a photon energy of the radiation, the control method comprising:
causing a computer to:
specify a high-sensitivity site in the subject, where sensitivity to the radiation is relatively higher than in other sites, based on an optical image of the subject acquired by a camera that acquires the optical image; and
set different parameters related to a dose of radiation to be emitted to the subject between the high-sensitivity site and the other sites.

11. A non-transitory computer-readable storage medium that stores a control program of a radiographic imaging apparatus including a photon-counting detector that detects radiation emitted from a radiation source and transmitted through a subject on a patient table and that outputs a detection signal corresponding to a photon energy of the radiation, the control program causing a computer to execute:
a procedure of specifying a high-sensitivity site in the subject, where sensitivity to the radiation is relatively higher than in other sites, based on an optical image of the subject acquired by a camera that acquires the optical image; and
a procedure of setting different parameters related to a dose of radiation to be emitted to the subject between the high-sensitivity site and the other sites.
